(19) United States
(12) Patent Application Publication
(10) Pub. No.: US 2010/0273844 A1
(43) Pub. Date: Oct. 28, 2010

(54) PHARMACEUTICAL COMPOSITIONS FOR TREATING OR PREVENTING MIGAINES

(75) Inventors: Richard Guarino, Verona, NJ (US); Eyal S. Ron, Lexington, MA (US)

Correspondence Address: Leslie G Restaino;Validus Pharmaceuticals Inc, 119 Cherry Hill Road Suite 310, Parsippany, NJ, 07054, US

(73) Assignee: OXFORD PHARMACEUTICAL SERVICES INC., Totawa, NJ (US)

(21) Appl. No.: 11/992,714

(22) PCT Filed: Sep. 27, 2006
(86) PCT No.: PCT/US2006/037882

§ 371 Date: Jul. 12, 2010

Related U.S. Application Data
(60) Provisional application No. 60/721,271, filed on Sep. 28, 2005.

(51) Int. Cl. A61K 31/42; C07D 261/18; A61P 25/06

(52) U.S. Cl. 514/378; 548/248

(57) ABSTRACT

The present invention provides a method for treating or preventing one or more types of migraines by administering a monoamine oxidase inhibitor, preferably isocarboxazid, in a rapid-release formulation. The present invention also provides a pharmaceutical composition suitable for treating or preventing migraines and having at least one monoamine oxidase inhibitor.

### RELATED APPLICATIONS

This application claims priority to provisional U.S. application No. 60/721,271, filed Sep. 28, 2005, the contents of which are incorporated by reference herein in their entirety.

### FIELD OF THE INVENTION

The present invention relates to pharmaceutical compositions containing monoamine oxidase inhibitors and methods of using such compositions for treating or preventing migraines.

### BACKGROUND OF THE INVENTION

Migraines are throbbing or pulsating headaches, often associated with nausea, vomiting, sensitivity to light, sleep disruption, and depression. According to the World Health Organization, migraines are one of the top 20 causes of years-of-life lived with disability. Migraines can be triggered by many factors, including lack of food, lack of sleep, exposure to light, hormonal irregularities (in women), anxiety, stress, or relaxation after stress.

Migraines are classified based on the symptoms they produce. The two most common types of migraines are migraine with aura and migraine without aura. Less common types include, but are not limited to, basilar artery migraine, carotidynia, headache-free migraine, opthalmoplegic migraine, and status migraine. See Report on the International Classification of Headache Disorders (2d Ed.), Cephalagia, 24 (1): 24-36 (2004).

A migraine attack can be divided into four distinct phases, plus the interval phase between different phases. The four stages are the prodromal phase, the aura phase, the headache phase, and the postdrome phase.

Physicians often adopt individualized approaches to treating acute migraines, usually based on the frequency of the headaches experienced by a patient. Preventative therapy is usually undertaken in patients who have more than two headache episodes per month and those patients who are disabled by headaches.

For many years, scientists believed that migraines were linked to the dilation and constriction of blood vessels in the head. Investigators now believe that migraines are caused by inherited abnormalities in genes that control the activities of certain cell populations in the brain.

Current migraine medications produce relief by quieting sensitive nerve pathways and reducing inflammation. The most frequently used agents for treating migraine are triptan agents. Other agents include dihydroergotamine, verapamil, diltiazem, dichloralphenazone, isometheptene, lisuride, lidocaine, cortisone, and various analgesics and NSAIDs. However, these agents and other currently available therapies frequently lack efficacy and have certain adverse effects. Therefore, there exists a need for a more effective migraine therapy without undesired adverse side effects.

Monoamine oxidase inhibitors (“MAOIs”) are a class of compounds which are commonly used in treating depression. They function by inhibiting the activities of monoamine oxidase thereby preventing degradation of monoamine neurotransmitters in the brain. It has been suggested that MAOIs may also have a beneficial effect against certain symptoms associated with migraines. See e.g., Claman J M, Proc Aust Assoc Neurol. 7:45-7 (1970) and Merikangas et al., Clin J Pain. 1989; 5(1):111-20 (1989). However, the use of MOAIs in treating migraines is not very well defined.

A substantial proportion of migraine patients have gastric stasis and suffer severe nausea and/or vomiting during their migraine attack. This may lead to erratic absorption from the gastrointestinal tract and make oral treatment unsatisfactory. For such patients, a non-oral formulation may be advantageous. Also, it is desirable to provide new dosage forms and new routes of administration to allow fast onset and reduce total dose exposure. An orally-absorbed form of one MAOI, selegiline, has been described in WO/1996/026720 for treatment of Parkinson's and Alzheimer's diseases.

Additionally, clinical use of MAOIs is often complicated by fast clearance from the bloodstream. Orally administered MAOIs are rapidly absorbed from the gastro-intestinal tract and have a short half-life, estimated to be 1-4 hours in humans. Because MAOIs are cleared from the body rapidly, their plasma drug levels often do not correlate with monoamine oxidase inhibition. Few studies on the pharmacokinetics and plasma drug levels of such MAOIs have been performed, despite several decades' clinical use of MAOIs in treating depression. As a result, MAOIs are administered twice or three times daily.

Two or three times daily administration is inconvenient for patients, especially those who must keep the MAOI blood level at a steady state. Frequent drug administration may reduce patient compliance. Such administration also produces irregular blood levels, and as a result, adverse effects can arise. Therefore, there is a need among migraine patients for a specifically designed formulation of MAOIs which effectively delivers MAOIs, while reducing dosing frequency.

### SUMMARY OF THE INVENTION

The present invention provides pharmaceutical compositions suitable for treating or preventing migraines, comprising an effective amount of an MAOI.

Another aspect of the present invention is a method of treating or preventing migraines in a subject in need thereof, comprising administering to the subject an effective amount of an MAOI, preferably isocarboxazid.

Yet another aspect of the present invention is a method of treating or alleviating symptoms caused by, or associated with migraines in a subject in need thereof, comprising administering to the subject an effective amount of an MAOI. These symptoms include, but are not limited to, nausea, photophobia, lightheadedness, scalp tenderness, vomiting, photopsia, fortification spectra, paresthesias, vertigo, syncope, seizure, confusional state and diarrhea.

Yet another aspect of the present invention is a method of treating or preventing various stages of migraine in a subject in need thereof, comprising administering to the subject an effective amount of an MAOI. These stages include, but are not limited to, the prodromal phase, the aura phase, the headache phase, and the postdrome phase.

A preferred embodiment of the present invention is a method of treating or preventing migraines in a subject in need thereof, comprising administering to the subject an effective amount of isocarboxazid, wherein the migraine is selected from a group consisting of migraine with aura, migraine without aura, basilar artery migraine, carotidynia, headache-free migraine, opthalmoplegic migraine, retinal migraine, familial hemiplegic migraine, abdominal migraine, acephalgic migraine, and status migraine.

Yet another aspect of the present invention is a method of administering isocarboxazid. In one embodiment, the method includes' administering isocarboxazid prophylactically. In another embodiment, the method includes administering isocarboxazid after onset of symptoms.

Yet another aspect of the present invention is a method of treating or preventing migraines accompanied by one or more other diseases, such as depression.

Yet another aspect of the present invention is a method for delivering an MAOI, preferably isocarboxazid, to a subject by oral, sublingual, buccal, nasal, interveinal, vaginal, topical or transdermal administration.

Yet another aspect of the present invention is a pharmaceutical composition suitable for treating or preventing migraines, comprising an effective amount of a MAOI other than selegiline, preferably isocarboxazid, in a fast-release formulation, which enables the release of isocarboxazid within 30 seconds of being placed in the oral cavity.

Yet another aspect of the present invention is a pharmaceutical composition suitable for treating or preventing migraines, comprising an effective amount of a MAOI other than selegiline, preferably isocarboxazid, in a fast-release formulation, which enables the release of isocarboxazid within 5 minutes of being administered sublingually or as a buccal dosage form

Yet another aspect of the present invention is a pharmaceutical composition suitable for treating or preventing migraines, comprising an effective amount of an MAOI, preferably isocarboxazid, in a fast-release formulation, which enables the release of isocarboxazid within 5 minutes of being administered intranasally.

Yet another aspect of the present invention is a pharmaceutical composition suitable for treating or preventing migraines, comprising an effective amount of isocarboxazid in a fast-release formulation, which enables the release of isocarboxazid within 5 minutes of being administered rectally or intravaginally.

Yet another aspect of the present invention is a two-component pharmaceutical composition suitable for treating or preventing migraine, comprising an effective amount of isocarboxazid in a fast-release form and also an effective amount of isocarboxazid in a sustained release form which enables the release of isocarboxazid for a time ranging from about 8 hours to about 48 hours after administration.

Yet another aspect of the present invention is a two-component pharmaceutical composition suitable for treating or preventing migraine, comprising an effective amount of isocarboxazid in a fast-release form and also an effective amount of isocarboxazid in a sustained release form which provides a substantially constant plasma level of isocarboxazid for a prolonged period of time ranging from about 8 hrs to about 48 hrs.

Yet another aspect of the present invention is a two-component pharmaceutical composition suitable for treating or preventing migraine, comprising an effective amount of isocarboxazid in a fast-release form and also an effective amount of isocarboxazid in a sustained release form which provides approximately 60% of the maximum plasma concentration of isocarboxazid within about 30 minutes after administration.

Other features and advantages of the present invention are described in the following detailed description. It should be understood, however, that the detailed description and the specific examples, while disclosing the preferred embodiments of the invention, are given by way of illustration only, and that various changes and modifications apparent to those skilled in the art are within the spirit and scope of the invention and the appended claims.

### DETAILED DESCRIPTION OF THE INVENTION

The term “effective amount,” as used herein, refers to a nontoxic but sufficient amount of the drug or pharmacologically active agent to provide the desired therapeutic effect. It is recognized that the effective amount of a drug or pharmacologically active agent will vary depending on the route of administration, the selected compound, and the subject species to which the drug or pharmacologically active agent is administered. It is also recognized that one of skill in the art will determine appropriate effective amounts by taking into account such factors as metabolism, bioavailability, and other factors that affect plasma levels of a drug or pharmacologically active agent following administration.

The term “controlled release,” as used herein, refers to the release of a drug from a drug-containing composition at a rate determined by the presence of a non-drug agent in the composition.

The term “sustained release,” as used herein, refers to a gradual release of a drug over an extended period of time, which preferably, although not necessarily, results in substantially constant blood levels of the drug over an extended time period. The extended period of time may be from 4 to 24 hours or longer in duration, preferably from 8 to 24 hours, and more preferably from 12 to 24 hours.

The term “fast release,” as used herein, refers to a drug formulation that provides for release of the drug immediately after drug administration.

The term “oral administration,” as used herein, refers to delivery of a drug through the mouth and ingestion through the stomach and digestive tract.

The term “transdermal,” as used herein, refers to delivery by passage of a drug through the skin or mucosal tissue and into the bloodstream.

The term “topical administration,” as used herein, refers to delivery of a topical drug or pharmacologically active agent to the skin or mucosa.

The term “subject,” as used herein, refers to both humans and animals.

The term “inhalation administration,” refers to delivery of an aerosolized form of the drug by passage through the nose or mouth during inhalation and passage of the drug through the walls of the nasal mucosal tissue and/or the lungs.

The term “intravesical administration,” as used herein, refers to delivery of a drug directly into the bladder.

The present invention provides a method of treating or preventing migraines in a subject in need thereof, by administering to the subject an effective amount of at least one MAOI. Suitable MAOIs include, but are not limited to, isocarboxazid, phenelzine sulfate, tranylcypromine, and their pharmaceutically acceptable salts. Additional examples of MAOIs include, but are not limited to deprenyl and moclobemide. Further examples of MAOIs can be found in Cesura et al., Prog. Drug Res. 38: 171-297 (1992).

In one preferred embodiment, the present invention provides a method of treating or preventing migraines in a subject in need thereof, comprising administering to the subject an effective amount of isocarboxazid or any of its pharmaceutically acceptable salts.

The present invention also provides methods of treating or alleviating one or more symptoms, caused by or associated with migraines, including nausea, photophobia, lightheadedness, scalp tenderness, vomiting, photopsia, fortification spectra, paresthesias, vertigo, syncope, seizure, confusion of mind state and diarrhea, by administering an effective amount of a MAOI. In particular, the present invention includes the use of isocarboxazid or any of its pharmaceutically acceptable salts in treating or alleviating symptoms caused by or associated with migraines.

The present invention also provides methods of treating or preventing different phases of migraine in a subject in need thereof, by administering an effective amount of a MAOI. Particularly preferred in the present invention is the use of isocarboxazid or any of its pharmaceutically acceptable salts.

The methods described in the present invention are useful for treating or preventing one or more types of migraines based on clinical symptoms. For example, the methods of the present invention may be used to treat or prevent migraine with aura, migraine without aura, basilar artery migraine, carotidynia, headache-free migraine, opthalmoplegic migraine, retinal migraine, familial hemiplegic migraine, abdominal migraine, acephalgic migraine, and/or status migraine. Further information on different types of migraine can be found in Harrison's Principles of Internal Medicine. See Raskin et al., Headache, Including Migraine and Cluster Headache, pages 73-39, In Braunwald et al. (ed.), Harrison's Principles of Internal Medicine (15ed), New York, McGraw Hill, Medical Publishing Division (2001).

In addition to migraines, the methods of the present invention are also useful for treating or preventing other diseases, such as depression, cluster headaches and trigeminal neuralgia. Thus, another embodiment of the present invention is a method of treating both migraines and another disease, such as depression and anxiety, in a subject in need thereof by administering an effective amount of at least one MAOI, preferable isocarboxazid.

The second aspect of the present invention is a pharmaceutical composition suitable for treating or preventing migraines comprising an effective amount of at least one MAOI or its pharmaceutically acceptable salt, and a pharmaceutically acceptable carrier. Among the preferred embodiments, the MAOI is isocarboxazid.

Another embodiment of the present invention is a pharmaceutical composition suitable for treating or preventing migraines, comprising an effective amount of at least one MAOI, preferably isocarboxazid, or its pharmaceutically acceptable salt, and a second therapeutic agent. Examples of the second therapeutic agent include, but are not limited to, dihydroergotamine, verapamil, diltiazem, dichloralphenazone, isometheptene, lisuride, lidocaine, cortisone, and various analgesics and NSAIDs such as acetaminophen, aspirin, ibuprofen, naproxen, nabumetone, and the like. Additional therapeutic agents useful for treating or alleviating symptoms associated with or caused by migraines may also be included.

MAOIs are well-known in the art, and many such compounds can be obtained commercially. For example, isocarboxazid is commercially available as MARPLAN® in a 10 mg tablet formulation, distributed by Oxford Pharmaceutical Services, Inc., Totowa, N.J. 07512.

The amount of a MAOI in a unit dose of such a pharmaceutical composition can range from about 0.01 mg to about 1000 mg, preferably from about 1 mg to about 500 mg, and most preferably from about 5 mg to about 100 mg. In a preferred embodiment, the concentration of an MAOI, ranges from about 10 mg to about 60 mg. In yet another preferred embodiment, the concentration of an MAOI is 5, 10, 20, 30, 40, 50, 60, 75 or 100 mg.

The third aspect of the present invention is a controlled-release formulation of a MAOI which is suitable for treating or preventing migraine. Examples of controlled-release formulations include, but are not limited to, fast release, sustained release, continuous, as needed, short-term, rapid-offset, delayed release, and pulsatile release formulations.

In one embodiment, the composition includes isocarboxazid and at least one controlled-release agent. In another embodiment, the controlled-release agent is a polymer that releases isocarboxazid by diffusion, in which isocarboxazid is encapsulated in a polymeric membrane or suspended within a polymer matrix. In another embodiment, the controlled-release agent is a semi-permeable membrane containing an osmotic agent that releases isocarboxazid by solvent activation or increased pressure. In yet another embodiment, the controlled-release agent is a degradable polymeric coat that degrades in a particular environment, for example, in a particular pH, and thereby releases isocarboxazid.

In a particular embodiment, isocarboxazid is incorporated in a controlled-release matrix. Examples of materials suitable for inclusion in a controlled-release matrix include one or more of water soluble polymers, water insoluble polymers, and fatty compounds. The term “water soluble polymer,” as used herein, includes polymers which can be dissolved in water. Examples of such water soluble polymers include, but are not limited to, Eudragit RL, polyvinyl alcohol, polyvinylpyrrolidone, methyl cellulose, hydroxypropyl cellulose, hydroxypropylmethyl cellulose, polyethylene glycol and mixtures thereof.

The term “water insoluble polymer,” as used herein, includes polymers which do not or only slightly dissolve in water. Examples of such water insoluble polymers include, but are not limited to, Eudragit RS, ethylcellulose, cellulose acetate, cellulose propionate (lower, medium or higher molecular weight), cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly(ethylene) high density, poly(propylene), poly(ethylene terephthalate), poly(vinyl isobutyl ether), poly(vinyl acetate), poly(vinyl chloride), polyurethane and a mixture thereof.

In one embodiment, the controlled-release matrix includes one or more water soluble polymers. In another embodiment, the controlled-release matrix includes one or more water insoluble polymers. In yet another embodiment, the controlled-release matrix includes a combination of one or more water soluble and one or more water insoluble polymers. In a particular embodiment, the controlled-release matrix includes a minor portion of one or more water insoluble polymers and a major portion of one or more water soluble polymers. In a certain embodiment, the controlled-release matrix includes a minor portion of one or more water soluble polymers and a major portion of one or more water insoluble polymers. The ratio of water soluble and water insoluble polymers may be determined by the particular combination of polymers selected. In certain embodiments, the controlled-release matrix includes about 10-50% of one or more hydrophilic polymers and about 10-50% of one or more hydrophobic polymers.

In some embodiments, the controlled-release matrix includes one or more fatty compounds. Examples of fatty compounds include, but are not limited to, waxes generally (e.g., carnauba wax) and glyceryl tristearate.

In a particular embodiment, the controlled-release matrix is designed to provide continuous and prolonged release of isocarboxazid over a period of from about 8 hours to about 24 hours. In one embodiment, the matrix provides the controlled release of isocarboxazid over at least an eight hour period. In another embodiment, the matrix provides the controlled release of isocarboxazid over at least a twelve hour period. In yet another embodiment, the matrix provides the controlled release of isocarboxazid over at least a twenty four hour period.

According to another embodiment, the isocarboxazid is incorporated in a matrix and the matrix is overcoated with a coat. In one embodiment, the coat is a fast-dissolving film, comprising one or more polymers. Examples of such polymers include, but are not limited to, polyvinylpyrrolidone, ethylcellulose, Eudragit RL, Eudragit L, Eudragit E, Eudragit S, cellulose acetate, polyvinyl alcohol, cellulose gum, Methocel E5, Pullalan (P-20), POLYOX WSR N-10, PVA (Vino) 125, and polypropylene glycol.

In another embodiment, the controlled-release coat includes one or more delayed release coating agents. Examples of delayed release coating agents include, but are not limited to, cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, polymers and copolymers formed from acrylic acid, methacrylic acid, and/or esters thereof.

In yet another embodiment, the controlled-release coat includes one or more sustained release coating agents. Sustained release dosage forms allow drug release over an extended time period. Examples of sustained release coating agents include, but are not limited to water insoluble polymers, water soluble polymers and fatty compounds as described above.

In certain embodiments, the controlled-release coat includes one or more hygroscopic agents. Examples of hygroscopic agents include, but are not limited to, microcrystalline cellulose (AVICEL PH 200, AVICEL PH 101), Ac-Di-Sol (Croscarmelose Sodium) and PVP-XL (a crosslinked polyvinylpyrrolidone), starches and modified starches, polymers, and gum such as arabic and xanthan, and hydroxyalkyl cellulose such as hydroxymethylcellulose, hydroxypropylcellulose and hydroxyopropylmethylcellulose.

In particular embodiments, the controlled-release coat may be made of commercially available ready-made polymeric solutions or suspensions. These ready made solutions or suspensions may optionally contain plasticizing agents to improve the coat. Examples of ready-made solutions or suspensions of polymeric material with or without plasticizing agent include, but are not limited to, Eudragit RL 30D, Eudragit L 30D, Euaragit E 12.5, Eudragit L 12.5 P, Eudragit E 12.5, Eudragit S 12.5P, Eudragit RL 12.5, Eudragit RS 12.5, (Eudragit being a Trade Mark of Rohm and Haas, whose technical brochures describe the differences between the products), Aquacoat (a Trade Mark of FMC Corporation) and Sure-lease (a Trade Mark of Colorcon Inc.).

Dosage forms with controlled-release coatings may be manufactured using standard coating procedures and equipment known to one skilled in the art. In some embodiments, after preparation of the solid dosage form, a controlled-release coating composition is applied using a coating pan, an airless spray technique, or fluidized bed coating equipment.

In one embodiment, the composition containing the coat completely disintegrates within about 30 seconds of being placed in the oral cavity. In another embodiment, the composition containing the coat completely disintegrates within about 10 to 25 seconds of being placed in the oral cavity.

The present invention also provides pharmaceutical compositions comprising at least an amount of isocarboxazid effective for the relief of migraine in a human subject, wherein the composition releases an effective amount of isocarboxazid into the bloodstream of the subject within about 5 minutes of administration, preferably within about 2 minutes of administration, and more preferably within about 1 minute of administration.

In certain embodiments, the composition containing the matrix and/or coat provides approximately 60% of the maximum plasma concentration (C-max) of isocarboxazid within about 10-45 minutes after administration. In a preferred embodiment, the composition containing the matrix and/or coat provides approximately 60% of the maximum plasma concentration (C-max) of the MAOI within about 30 minutes after administration (T-max).

In a particular embodiment, the composition of the present invention provides a release of approximately 15% of the contained isocarboxazid after about 2 hours, between approximately 20% and approximately 60% of the isocarboxazid after about 8 hours and greater than approximately 65% of the isocarboxazid after about 12 hours. In some embodiments, the composition releases approximately 90% or more of the isocarboxazid after about 20 hours.

The potential for oral absorption of compositions containing MAOIs can be assessed using the method described in PCT International Patent Application No. WO/1996/026720 and by Harris and Robinson in J. Pharm. Sci. 81:1-10 (1992). Essentially, a test formulation containing the clinically effective dose of the MAOI is retained in the mouth for 1 minute before it is expectorated. The mouth is then rinsed with water, and the total amount of MAOI in the expectorated mouth washings is determined using a suitable analytical technique such as HPLC. The recovered quantity of MAOI is subtracted from the total amount of drug initially placed in the mouth to determine the total amount of drug which has been absorbed. For significant oral absorption to have occurred it is generally preferred that at least 5% of the MAOI has been absorbed in 1 minute in this test, more preferably that at least 10% has been absorbed in 1 minute and most preferably at least 15% of the MAOI has been absorbed in 1 minute.

It is envisaged that oral absorption will occur primarily across the mucous membranes in the mouth, pharynx and oesophagus. Accordingly, it is preferred that the composition of the invention is adapted to promote contact of the active ingredient with the buccal, sublingual, pharyngeal and/or oesophageal mucous membranes, as is known in the art.

Suitable compositions include but are not limited to viscous emulsions, syrups or elixirs, sub-lingual tablets, suckable or chewable tablets or candies, softgels, lozenges, aqueous or non-aqueous drops, bioadherent systems, or other dosage forms designed to release the active ingredient in a controlled manner to saliva or to the buccal, pharyngeal and/or oesophageal mucous membranes. Particularly preferred are fast-dispersing dosage forms designed to release the active ingredient rapidly in the oral cavity.

Examples of bioadherent systems may be found in U.S. Pat. No. 5,055,303. Another example of a fast-dispersing dosage form is described in U.S. Pat. No. 4,855,326 in which a melt spinnable carrier agent, such as sugar, is combined with an active ingredient and the resulting mixture spun into a cotton candy-like compositon, which is then compressed into a rapidly dispersing, highly porous solid dosage form. U.S. Pat. No. 5,120,549 discloses a fast-dispersing matrix system which is prepared by first solidifying a matrix-forming system dispersed in a first solvent and subsequently contacting the solidified matrix with a second solvent that is substantially miscible with the first solvent at a temperature lower than the solidification point of the first solvent, the matrix-forming elements and active ingredient being substantially insoluble in the second solvent, whereby the first solvent is substantially removed resulting in a fast-dispersing matrix.

U.S. Pat. No. 5,079,018 discloses a fast-dispersing dosage form which comprises a porous skeletal structure of a water soluble, hydratable gel or foam forming material that has been hydrated with water, rigidified in the hydrated state with a rigidifying agent and dehydrated with a liquid organic solvent at low temperature.

Published International Application No. WO 93/12769 describes fast-dispersing dosage forms of very low density formed by gelling, with agar, aqueous systems containing the matrix-forming elements and active ingredient, and then removing water by forced air or vacuum drying. U.S. Pat. No. 5,298,261 discloses fast-dispersing dosage forms which comprise a partially collapsed matrix network that has been vacuum-dried above the collapse temperature of the matrix. International Application No. WO 91/04757 discloses fast-dispersing dosage forms which contain an effervescent disintegration agent designed to effervesce on contact with saliva to provide rapid disintegration of the dosage form and dispersion of the active ingredient in the oral cavity. U.K. Patent No. 1548022 discloses a solid fast-dispersing dosage form comprising a network of the active ingredient and a water-soluble or water-dispersible carrier, obtained by subliming solvent from a frozen composition.

The composition of the invention may also be in the form of granules, spheroids, pellets, multiparticulates, capsules, sachets, controlled-release suspensions, gels, aerosols or any other suitable dosage form known to one skilled in the art. In some embodiments, the pH of the liquid or aerosol composition ranges from about 5.0 to about 8.0. In a particular embodiment, the pH of the composition is 5.4.

In some embodiments, the composition of the invention includes other materials such as binders, diluents, lubricants, disintegrants, fillers, stabilizers, surfactants, preservatives, coloring agents, and/or flavoring agents.

Binders are used to impart cohesive qualities to a tablet composition, and ensure that the tablet remains intact after compression. Examples of binders include, but are not limited to, starch (including corn starch and pregelatinized starch), gelatin, sugars (including sucrose, glucose, dextrose and lactose), polyethylene glycol, propylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, ethyl cellulose, hydroxyethyl cellulose, and the like), and Veegum.

Diluents are typically employed to increase the bulk characteristics of the composition. Examples of diluents include, but are not limited to, dicalcium phosphate, calcium sulfate, lactose, cellulose, kaolin, mannitol, sodium chloride, dry starch and powdered sugar.

Lubricants are used to facilitate the manufacturing process. Examples of lubricants include, but are not limited to, vegetable oils such as peanut oil, cottonseed oil, sesame oil, olive oil, corn oil, and oil of theobroma, glycerin, magnesium stearate, calcium stearate, and stearic acid.

Disintegrants are used to facilitate disintegration of the composition. Examples of disintegrants include, but are not limited to, starches, clays, celluloses, algins, gums and crosslinked polymers.

Examples of fillers include, but are not limited to, silicon dioxide, titanium dioxide, alumina, talc, kaolin, powdered cellulose and microcrystalline cellulose, as well as soluble materials such as mannitol, urea, sucrose, lactose, dextrose, sodium chloride and sorbitol.

Stabilizers are used to inhibit or retard drug decomposition reactions such as oxidative degradation. Surfactants may be anionic, cationic, amphoteric or nonionic surface active agents.

Examples of other classes of additives include, but are not limited to, hydrocolloid suspending agents, buffering agents, stabilizers, foaming agents, pigments, coloring agents, fillers, bulking agents, sweetening agents, flavoring agents, fragrances, and release modifiers.

The composition of the present invention may be delivered orally, mucosally, intravenously, intravesically, intrathecally, intravaginally, rectally, topically, transdermally, nasally, by inhalation, through implantable systems or any other route of administration known to one skilled in the art. The orally administered composition may be in the form of a solid or a liquid. Oral dosage forms include tablets, capsules, caplets, solutions, suspensions and/or syrups, and may also comprise a plurality of granules, beads, powders or pellets that may or may not be encapsulated. Such dosage forms are prepared using methods known to one skilled in the art.

Examples of mucosal administration include buccal, sublingual, intranasal, transurethral, rectal, and vaginal administration. Nasal administration includes, for example, intranasal sprays and inhalers. Transdermal administration may be in the form for example of transdermal patches, gels, or foams.

The compositions adapted for rapid buccal and/or sublingual administration may, in some embodiments, further comprise a sustained-release component for oral administration of isocarboxazid. The sustained-release component releases isocarboxazid at a rate that maintains in the subject a blood level of isocarboxazid effective to treat or prevent migraine for at least 8 hours, preferably for at least 12 hours, and most preferably for at least 24 hours.

By way of example, a sustained-release tablet manufactured by any of the sustained-release technologies known in the art is coated with a fast-dissolving layer comprising an amount of isocarboxazid effective for relief of migraine. The amount of isocarboxazid in the fast-dissolving coating may be, for example, 5, 10, or 15 mg. Administration of the tablet involves holding the tablet in the mouth, preferably under the tongue, for a period of time sufficient for dissolution of the coating and absorption of an effective amount of isocarboxazid through the oral mucosa. The remainder of the tablet is then swallowed.

Nasal dosage forms may be administered intranasally or by inhalation. In some embodiments, the compositions for intranasal administration are liquid formulations for administration as a spray or in the form of drops. For liquid formulations, the active agent can be formulated into a solution, e.g., water or isotonic saline, buffered or unbuffered, or as a suspension. In certain embodiments, such solutions or suspensions are isotonic relative to nasal secretions and of about the same pH, ranging e.g., from about pH 4.0 to about pH 7.4 or, from about pH 6.0 to about pH 7.0. In one embodiment, the buffer is a phosphate buffer.

In some embodiments, formulations for inhalation may be prepared as an aerosol, either a solution aerosol in which the active agent is solubilized in a carrier, such as a propellant, or a dispersion aerosol, in which the active agent is suspended or dispersed throughout a carrier and an optional solvent. Non-aerosol formulations for inhalation may take the form of a liquid, for example, an aqueous suspension or aqueous solution.

The devices for delivering liquid formulations are known to one skilled in the art. Such devices may generate drops, droplets and sprays. Examples of such devices include, but are not limited to, droppers, squeeze bottles, and manually and electrically powered intranasal pump dispensers.

In other embodiments, the compositions for intranasal administration are powder formulations, for example, nasal gels, creams, pastes or ointments. In some embodiments, the viscosity of the formulation may range from about 10 to about 6500 cps, depending on the desired sustained contact with the nasal mucosal surfaces. Intranasal formulations may include carriers known to one skilled in the art. Examples of such carriers include alkylcelluloses and/or other biocompatible carriers of high viscosity well known to the art. Intranasal formulations may include other ingredients, such as, preservatives, colorants, lubricating or viscous mineral or vegetable oils, perfumes, natural or synthetic plant extracts such as aromatic oils, and humectants and viscosity enhancers (e.g., glycerol).

Pharmaceutical compositions of the invention can be prepared in a formulation suitable for rectal administration, by methods well-known in the art. Such a composition can be in the form of, for example, a suppository, a retention enema preparation, and a solution for rectal or colonic irrigation. Suppository formulations can be made by combining the monoamine oxidase inhibitor with a non-irritating pharmaceutically acceptable excipient which is solid at ordinary room temperature and which is liquid at the rectal temperature of the subject (about 37 C in a healthy human). Suitable pharmaceutically acceptable excipients include, but are not limited to, cocoa butter, polyethylene glycols, and various glycerides. Retention enema preparations or solutions for rectal or colonic irrigation can be made by combining the active agent with a pharmaceutically acceptable liquid carrier. As is well known in the art, enema preparations can be administered using, and can be packaged within, a delivery device adapted to the rectal anatomy of the subject.

Pharmaceutical compositions of the invention can also be prepared in a formulation suitable for vaginal administration, by methods well-known in the art. Such a composition can be in the form of, for example, a suppository, an impregnated or coated vaginally-insertable device such as a tampon, a douche preparation, or a solution for vaginal irrigation. Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying. Douche preparations or solutions for vaginal irrigation can be made by combining the active agent with a pharmaceutically acceptable liquid carrier. As is well known in the art, douche preparations can be administered using, and can be packaged within, a delivery device adapted to the vaginal anatomy of the subject.

Preparations for rectal or vaginal application may further comprise various additional ingredients, including but not limited to antioxidants, antibiotics, anti-fungal agents, and preservatives.

The following examples are provided to further illustrate the invention described herein.

### EXAMPLES

### Example 1

### Effect of Isocarboxazid in Treating Migraine with Aura

A double blind, multicenter, randomized, placebo-controlled study is performed to evaluate the efficacy of isocarboxazid in treating patients suffering from migraine with aura.

A total of 16 patients participate in the study in a single center. The age of the recruited patients ranges between 18 years and 75 years. Both male and female patients are selected for the study. Recruitment period for the study is 5 months.

Isocarboxazid (Marplan®) 10 mg tablets are the oral medications used in the study. The dosage of isocarboxazid tablets ranges from 10 mg (1 tablet) to 60 mg (6 tablets) per day.

Isocarboxazid is prescribed as a prophylaxis to one group of patients. Also, isocarboxazid is prescribed after the expression of symptoms to another group of patients. The starting dose is 20 mg of isocarboxazid per day (10 mg tablets taken twice a day). Dose increments are based on the investigator's judgment of the patient's response. After 4 weeks of therapy on 20 mg per day, the doses may be increased to 30 mg per day, 10 mg in the AM and 20 mg in the PM. After 4 weeks of therapy on 30 mg per day, doses may be increased to 40 mg per day, 20 mg in the AM and 20 mg in the PM. After 4 weeks of 40 mg per day, the doses may be increased to 50 mg per day, 20 mg in the AM and 30 mg in the PM. After 4 weeks of 50 mg per day, the doses may be increased to 60 mg per day, 30 mg in the AM and 30 mg in the PM. The duration of the medication treatment is a total of 20 weeks.

Patients in the study are diagnosed for the symptoms of migraine with aura. Diagnosis is performed according to the criteria set forth by the International Headache Society. See Report on the International Classification of Headache Disorders (2d Ed.), Cephalagia, 24 (1): 24-36 (2004).

In order to determine the efficacy of isocarboxazid, pain intensity is evaluated on an assessment scale and recoded in an assessment sheet. Additionally, nausea, photophobia, phonophobia, and incidence of migraine attack are binary efficacy variables collected on the same assessment sheet.

The collected data are statistically analyzed. It is expected that isocarboxazid at a dosage ranging from 10 mg to 40 mg per day will have a superior effect over the placebo in treating and preventing migraine with aura.

### Example 2

### Effect of Isocarboxazid in Treating Migraine without Aura

A double blind, multicenter, randomized, placebo-controlled study is performed to evaluate the efficacy of isocarboxazid in treating patients suffering from migraine without aura.

A total of 16 patients participate in the study in a single center. The age of the recruited patients ranges between 18 years and 75 years. Both male and female patients are selected for the study. Recruitment period for the study is 5 months.

Isocarboxazid (Marplan®) 10 mg tablets are the oral medications used in the study. The dosage of isocarboxazid tablets ranges from 10 mg (1 tablet) to 60 mg (6 tablets) per day.

Isocarboxazid is prescribed as a prophylaxis to one group of patients. Also, isocarboxazid is prescribed after the expression of symptoms to another group of patients. The starting dose is 20 mg of isocarboxazid per day (10 mg tablets taken twice a day). Dose increments are based on the investigator's judgment of the patient's response. After 4 weeks of therapy on 20 mg per day, the doses may be increased to 30 mg per day, 10 mg in the AM and 20 mg in the PM. After 4 weeks of therapy on 30 mg per day, doses may be increased to 40 mg per day, 20 mg in the AM and 20 mg in the PM. After 4 weeks of 40 mg per day, the doses may be increased to 50 mg per day, 20 mg in the AM and 30 mg in the PM. After 4 weeks of 50 mg per day, the doses may be increased to 60 mg per day, 30 mg in the AM and 30 mg in the PM. The duration of the medication treatment is a total of 20 weeks.

Patients in the study are diagnosed for the symptoms of migraine without aura. Diagnosis is performed according to the criteria set forth by the International Headache Society. See Report on the International Classification of Headache Disorders (2d Ed.), Cephalagia, 24 (1): 24-36 (2004).

In order to determine the efficacy of isocarboxazid, pain intensity is evaluated on an assessment scale and recoded in an assessment sheet. Additionally, nausea, photophobia, phonophobia, and incidence of migraine attack are binary efficacy variables collected on the same assessment sheet.

The collected data are statistically analyzed. It is expected that isocarboxazid at a dosage ranging from 10 mg to 40 mg per day will have a superior effect over the placebo in treating and preventing migraine without aura.

### Example 3

### Effect of 20 mg and 40 mg Doses of Isocarboxazid in the Acute Treatment of Migraine

A double blind, multicenter, randomized, placebo-controlled single dose study to evaluate the efficacy of isocarboxazid in the treatment of acute migraine headaches is conducted.

This study has the objective of demonstrating the superiority of 20 mg and 40 mg doses of isocarboxazid over a placebo in the acute treatment of migraine headaches. The study consists of a screening visit, at home treatment of a single migraine attack, and a follow up visit 1-5 days following treatment. In addition to safety evaluations, the patients are evaluated for efficacy based on superiority over the placebo at 2 hour and 24 hour periods after dosing for a) pain relief; b) incidence of photophobia; c) incidence of phonophobia; and d) incidence of nausea.

The results are collected from 30 patients. It is expected that 20 and 40 mg doses of isocarboxazid will have a superior effect over a placebo after 2 hrs and 24 hours in treating acute headaches.

### Example 4

### Effect of Isocarboxazid on Migraine and Chronic Tension-Type Headache

This study is designed to evaluate the efficacy of isocarboxazid in preventing migraines and in the treatment of chronic tension-type headache.

A total of 61 patients suffering from chronic tension-type headache are recruited. The patients are classified according to the diagnostic criteria of the International Headache Society, and are treated for about 8 months on average with isocarboxazid (10 to 60 mg daily dose). While on this therapy, 35 of the 42 migraine patients and 16 of the 17 patients with tension-type headaches are experiencing good or very good improvement in their symptoms. In the migraine patients, the average number of monthly headache days is expected to decline at the end of treatment. In tension-type headaches, the effect may occur at the earliest 3-6 weeks and in the case of migraines at the earliest 6-8 weeks after the start of treatment. The therapeutic result is independent of any concurrent depression.

### Example 5

### Fast Dissolve Film Containing Isocarboxazid

Fast dissolve films are prepared as follows: a homogeneous mixture of ingredients is prepared in a coating solution in the amounts indicated in Table 1.

TABLE 1
Formulation of fast dissolving films

| Composition: coating solution | | | |
|---|---|---|---|
| (%) | Example 4a | Example 4b | Example 4c |
| isocarboxazid (%) | 10 | 10 | 10 |
| Pullalan (P-20) w % | | 17.5 | |
| Methocel E5 w % | 21.06 | | |
| POLYOX WSR N-10 w % | | | 1.8 |
| PVA (Vino) 125) w % | | 1.5 | |
| Cellulose gum w % | | | 8.1 |
| Propylene glycol w % | 1.0 | | 2.5 |
| Aspartame w % | 0.8 | 0.5 | 0.46 |
| Peppermint w % | 1.0 | 1.0 | 0.6 |
| Citric acid w % | 0.7 | 0.8 | |
| Cremphor EL40 w % | | 1.0 | 1.0 |
| Benzoic acid w % | 0.013 | 0.1 | 0.01 |
| Ethanol w % | | 10.6 | |
| Water w % | 64.42 | 57.0 | 75.6 |

TABLE 2
Properties of the film that are formed from the solutions of Table 1.

| Properties of dry film | Example 1 | Example 2 | Example 3 |
|---|---|---|---|
| Thickness (mm) | 0.6 | 0.7 | 0.7 |
| Disintegration (sec) | 12 | 20 | 12 |
| Dissolving time (sec) | 41 | 60 | 39 |

### Example 6

### Sublingual Spray

1200 mg of citric acid is dissolved in 20 ml of deionized water. 3 g of isocarboxazid is added to the solution and agitation is applied up to reaching a complete dissolution. 200 mg of propyleneglycol are added while maintaining the agitation. Subsequently the pH is adjusted to 5.4. Deionized water was added in an amount enough to bring the formulation to 100 g and the resulting solution is filtered through a 0.22 μm filter. The resulting solution is sprayed sublingually.

### Example 7

### Fast Dissolving Tablet

The following is a suitable formulation for a 10 mg isocarboxazid fast dissolve tablets:

Formula:

| | | |
|---|---|---|
| Isocarboxazid | 10 | mg |
| Powdered Mannitol | 425 | mg |
| Citric Acid | 11 | mg |
| Sweetener | 30 | mg |
| Glidant | 2 | mg |
| Lubricant | 9.75 | mg |
| Hygroscopic Agent | 52 | mg |
| Flavor | 22.75 | mg |
| Color | 1.95 | mg |
| Total | 565 | mg total tablet weight |

Hygroscopic agents include, for example, microcrystalline cellulose (AVICEL PH 200, AVICEL PH 101), Ac-Di-Sol (Croscarmelose Sodium) and PVP-XL (a crosslinked polyvinylpyrrolidone); starches and modified starches, polymers, and gum such as arabic and xanthan, and hydroxyalkyl celluloses such as hydroxymethylcellulose, hydroxypropylcellulose and hydroxypropylmethylcellulose.

Tablets are produced using a direct compression method as follows: All of the material, except the lubricant are weighed and blended. Thereafter, the lubricant is added and the mixture is blended. The blend is then tableted on a conventional tablet press. The average in vitro disintegration time is less than 30 seconds. The result may produce a disintegrating tablet and a pleasant organoleptic experience for the patients.

### Example 8

### Fast Release Coat and Sustained Release Core

Polyvinylpyrrolidone K-30 (2 kg) is dissolved in isopropylalcohol (10 kg). Isocarboxazid (1 kg) is added to this solution and allowed to dissolve. Carboxymethylcellulose (3 kg) is added to 9.75 Kg of the above solution and the solvent is evaporated. The resulting powder is then granulated to obtain fine particles. The powder (30%) is formed into a tablet with Methocel K4M 8.0%, Avicel pH 101 61.5% and magnesium stearate 0.5%. The tablet is coated with 3.25 Kg of the above remaining solution. The resulting product forms a fast release coat with a sustained release core.

### Example 9

### Intranasal Formulation

The effectiveness of the intranasal formulation of isocarboxazid (10 mg) is studied in a pilot clinical study. After intranasal administration, isocarboxazid may be directly and rapidly absorbed, with 60% of the maximum plasma concentration (Cmax) occurring at 30 minutes after administration of a single 10 mg dose. Following intranasal administration, approximately 10% more isocarboxazid may be absorbed as compared to the amount absorbed following oral administration. The mean Cmax after a 10 mg intranasal dose may be approximately 15 ng/mL, with the median time to Cmax being approximately 1 hour. When given as a single dose, intranasal isocarboxazid may display dose proportionality in its extent of absorption and a Cmax over the dose range 5 to 10 mg, but not between 5 and 20 mg for Cmax. The elimination phase half-life may be approximately 2 hours, consistent with administration by other routes.

1. A pharmaceutical composition comprising at least an amount of a monoamine oxidase inhibitor effective for the relief of migraine in a human subject, wherein the composition releases an effective amount of monoamine oxidase inhibitor into the bloodstream of the subject within about 5 minutes of administration, with the proviso that the monoamine oxidase inhibitor is not selegiline.

2. The composition according to claim 1, wherein the composition releases an effective amount of monoamine oxidase inhibitor into the blood stream of the subject within about 2 minutes of administration.

3. The composition according to claim 2, wherein the composition releases and effective amount of monoamine oxidase inhibitor into the blood stream of the subject within about 1 minute of administration.

4. A composition according to claim 1, wherein the composition is adapted for intranasal administration.

5. A composition according to claim 1, wherein the composition is adapted for buccal or sublingual administration.

6. A composition according to claim 1, wherein the composition is adapted for pulmonary administration.

7. A composition according to claim 5, wherein the composition further comprises a sustained-release component, said sustained-release component comprising a monoamine oxidase inhibitor and adapted for oral administration.

8. The composition according to claim 7, wherein said sustained-release component releases monoamine oxidase inhibitor at a rate that maintains a blood level of monoamine oxidase inhibitor effective to treat or prevent migraine for at least 8 hours.

9. The composition according to claim 8, wherein said sustained-release component releases monoamine oxidase inhibitor at a rate that maintains a blood level of monoamine oxidase inhibitor effective to treat or prevent migraine for at least 12 hours.

10. The composition according to claim 9, wherein said sustained-release component releases monoamine oxidase inhibitor at a rate that maintains a blood level of monoamine oxidase inhibitor effective to treat or prevent migraine for at least 24 hours.

11. A pharmaceutical composition comprising at least an amount of isocarboxazid effective for the relief of migraine in a human subject, wherein the composition releases an effective amount of isocarboxazid into the bloodstream of the subject within about 5 minutes of administration.

12. The composition according to claim 11, wherein the composition releases an effective amount of isocarboxazid into the bloodstream of the subject within about 2 minutes of administration.

13. The composition according to claim 12, wherein the composition releases an effective amount of isocarboxazid into the bloodstream of the subject within about 1 minute of administration.

14. A composition according to claim 11, wherein the composition is adapted for intranasal administration.

15. A composition according to claim 11, wherein the composition is adapted for buccal or sublingual administration.

16. A composition according to claim 11, wherein the composition is adapted for pulmonary administration.

17. A composition according to claim 15, wherein the composition further comprises a sustained-release component, said sustained-release component comprising isocarboxazxid and adapted for oral administration.

18. The composition according to claim 17, wherein said sustained-release component releases isocarboxazid at a rate that maintains a blood level of isocarboxazid effective to treat or prevent migraine for at least 8 hours.

19. The composition according to claim 18, wherein the sustained-release component releases isocarboxazid at a rate that maintains a blood level of isocarboxazid effective to treat or prevent migraine for at least 12 hours.

20. The composition according to claim 19, wherein the sustained-release component releases isocarboxazid at a rate that maintains a blood level of isocarboxazid effective to treat or prevent migraine for at least 24 hours.

21. A method for the prevention or relief of migraine in a human subject, comprising administration to the subject of a composition according to claim 1.

22. A method for the prevention or relief of migraine in a human subject, comprising administration to the subject of a composition according to claim 4.

23. A method for the prevention or relief of migraine in a human subject, comprising administration to the subject of a composition according to claim 5.

24. A method for the prevention or relief of migraine in a human subject, comprising administration to the subject of a composition according to claim 6.

25. A method for the prevention or relief of migraine in a human subject, comprising administration to the subject of a composition according to claim 7.

26. A method for the prevention or relief of migraine in a human subject, comprising administration to the subject, of a composition according to claim 11.

27. A method for the prevention or relief of migraine in a human subject, comprising administration to the subject of a composition according to claim 14.

28. A method for the prevention or relief of migraine in a human subject, comprising administration to the subject of a composition according to claim 15.

29. A method for the prevention or relief of migraine in a human subject, comprising administration to the subject of a composition according to claim 16.

30. A method for the prevention or relief of migraine in a human subject, comprising administration to the subject of a composition according to claim 17.

31. A method for the prevention or relief of migraine in a human subject, comprising administration to the subject of a composition comprising at least an amount of a monoamine oxidase inhibitor effective for the relief of migraine in a human subject, wherein the composition releases an effective amount of monoamine oxidase inhibitor into the bloodstream of the subject within about 5 minutes of administration.
